(19) United States
(12) Patent Application Publication
(10) Pub. No.: US 2026/0305991 A1
(43) Pub. Date: Oct. 8, 2026

(54) SYSTEM AND METHOD FOR HYPERSPECTRAL IMAGING AND MAPPING OF TISSUE OXYGEN SATURATION

(71) Applicant: Covidien LP, Mansfield, MA (US)

(72) Inventors: Soren AASMUL, Copenhagen (DK); Matthew S. ESCHBACH, North Haven, CT (US); Robert H. KNAPP, North Haven, CT (US); Bruno NASCIMENTO MENEZES, Copenhagen (DK)

(73) Assignee: Covidien LP, Mansfield, MA (US)

(21) Appl. No.: 19/478,922

(22) PCT Filed: Apr. 10, 2024
(86) PCT No.: PCT/US2024/023803

§ 371 Date: Oct. 27, 2025

Related U.S. Application Data
(60) Provisional application No. 63/462,295, filed on Apr. 27, 2023.
(60) Provisional application No. 63/568,532, filed on Mar. 22, 2024.

(51) Int. Cl. A61B 5/1455; A61B 1/00; A61B 5/1459; A61B 5/1495

(52) U.S. Cl. CPC A61B 5/14552; A61B 5/1459; A61B 5/1495; A61B 1/00009

(57) ABSTRACT

An imaging system includes a light source configured to output white light, a first wavelength light primarily absorbed by deoxyhemoglobin, and a second wavelength light primarily absorbed by oxyhemoglobin to illuminate tissue. The system further includes one or more cameras coupled to the scope. One or more cameras are configured to capture a plurality of frames of a tissue illuminated by the white light, the first wavelength light, and the second wavelength light. The system also includes an image processing unit configured to activate the light source to output the first wavelength light; receive a first single wavelength image; activate the light source to output the second wavelength light; receive a second single wavelength image; and generate a hyperspectral image based on the first single wavelength image and the second single wavelength image.

### CROSS-REFERENCE TO RELATED APPLICATIONS

This application claims the benefit of, and priority to, U.S. Provisional Patent Application Ser. No. 63/462,295 filed on Apr. 27, 2023, and U.S. Provisional Patent Application Ser. No. 63/568,532 filed on Mar. 22, 2024. The entire contents of the foregoing applications are incorporated by reference herein.

### BACKGROUND

Sufficient perfusion is essential to proper healing following a colorectal anastomosis procedure. The colon tissue may have compromised perfusion due to a number of reasons. One reason is a larger part of blood supply to the colon requires to be transected in connection with the anastomosis procedure. This leaves the colon with one or more regions with no or compromised blood supply. Locating the anastomosis in a region with poor or no blood supply increases the risk of a leak, which may also result in sepsis.

Conventional perfusion techniques primarily rely on fluorescence imaging, which requires infusion of indocyanine green contrast agent in the blood stream and comparing relative infrared intensities between perfused and non-perfused areas.

### SUMMARY

The present disclosure provides a system and method for real-time multispectral camera-based mapping of oxygen saturation and perfusion in tissue, e.g., colon tissue. Multispectral imaging includes capturing images of a target (e.g., tissue) that is illuminated with light at different, i.e., two or more, wavelengths.

The system includes a camera, which may be any suitable camera, e.g., laparoscopic or open camera, configured for video and/or still image capture. The camera may include an image sensor e.g., complementary metal oxide semiconductor (CMOS) or charge-coupled device (CCD) sensor, which is sensitive in the 350-1050 nm region or an InGaAs sensor, which is sensitive in the shortwave infrared (SWIR) region of 900-1700 nm. The sensor may be a color, or RGB, sensor or a monochrome sensor without an infrared filter. The camera also includes a plurality of controllable light sources, which are capable of selectively emitting a number of wavelengths specific to the oxy and deoxyhemoglobin absorption spectra. The light sources may include a plurality of LEDs configured to output light at specific wavelengths, which may be from about 380 nm to about 1,000 nm when using a CMOS or CCD sensor, e.g., 540 nm, 560 nm, 580 nm, 660 nm, 720 nm, 770 nm, 810 nm, 860 nm, and 940 nm, or 900 to about 1700 nm when using a InGaAs sensor, e.g., 1020 nm, 1040 nm, 1070 nm, 1200 nm, 1300 nm, 1450 nm, and 1550 nm. In embodiments, white light (i.e., combined visible spectrum) may also be emitted for normal imaging.

During video recording, light at each of the selected wavelengths is emitted sequentially with only one wavelength emitted and synchronized per frame of the camera. Other frames captured by the camera may be illuminated with white light to provide normal imaging. The wavelength specific frames may be omitted from the live view output on a monitor for viewing by a surgeon, and a previous white light frame may be used to fill in for the omitted multispectral light frame.

On a pixel level, the intensity ratio between frames illuminated with light at different wavelengths may be calculated and processed using an algorithm to map out tissue oxygen saturation. In embodiments, a ratio between 660 nm and 960 nm may be used to map out oxygen saturation in tissue. The result may be shown to the surgeon as a false color image, grey scale image, or in any other manner to differentiate with white color images. In embodiments, the image representing the oxygen saturation may be overlaid the white light image.

In further embodiments, prior to applying the intensity calculation algorithm, the frame may be compensated to improve the uniformity of the mapping to reduce vignetting caused by un-even illumination. Compensation may be performed by applying the inverse of the intensity distribution across the frame as recorded on a spectrally white reference target, which may be provided during calibration of the camera. If ambient light reaches the camera together with the selected single wavelength, compensation adds an offset to the detected intensity. The compensation will affect the ratio between the images formed by first and second wavelength. A mitigation step may also be used to record an image without any light source turned on and then subsequently subtract from frames before compensating for uneven illumination. In this way ambient light will be cancelled out.

In addition to monitoring tissue oxygenation, the imaging system may also be used to image other compounds and parameters of the tissue, e.g., water content, lipids (e.g., triglycerides), collagen, etc.

According to one embodiment of the present disclosure, an imaging system is disclosed. The imaging system includes a light source configured to output white light, a first wavelength light, and a second wavelength to illuminate tissue. The absorption spectrum of hemoglobin present in the tissue and in particular the absorption at the two said wavelengths varies with the oxygenation of the hemoglobin. The system also includes a scope configured to receive the white light, the first wavelength light, and the second wavelength light reflected from the tissue. The system further includes one or more cameras coupled to the scope. One or more cameras are configured to capture a plurality of frames of a tissue illuminated by the white light, the first wavelength light, and the second wavelength light. The system also includes an image processing unit configured to activate the light source to output the first wavelength light; receive a first single wavelength image of the tissue illuminated by the first wavelength light; activate the light source to output the second wavelength light; receive a second single wavelength image of the tissue illuminated by the second wavelength light; and generate a multispectral image based on the first single wavelength image and the second single wavelength image.

Implementations of the above embodiment may include one or more of the following features. According to one aspect of the above embodiment, the image processing unit may be further configured to activate the light emitter intermittently to emit the first wavelength light and the second wavelength light interspersed with the white light. The image processing unit may be also configured to replace the first single wavelength image and the second single wavelength image with a preceding white light image from the plurality of frames and generate a processed video feed. The imaging system may additionally include a monitor coupled to the image processing unit and configured to display the processed video feed. The image processing unit may be further configured to generate an intensity ratio image based on the first single wavelength image and the second single wavelength image. The image processing unit may be further configured to generate the intensity ratio image by calculating an intensity ratio on a pixel-by-pixel ratio between the first single wavelength image and the second single wavelength image. The image processing unit may be additionally configured to generate a multispectral image by colorizing the intensity ratio image. The image processing unit may be further configured to overlay the multispectral image over the processed video feed. The light emitter may include a plurality of light emitting diodes (LED) having a first LED configured to emit the first wavelength light, a second LED configured to emit the second wavelength light, and a third LED configured to emit the white light. The light source may be configured to output and transmit the first wavelength light, the second wavelength light, and the white light to the light emitter. The light emitter may include a laser diode or white light filtered by a monochromator or di-electrical filters or a white-light continuum light source (e.g., a “white laser”).

According to another embodiment of the present disclosure, a method for imaging perfusion of tissue is disclosed. The method includes activating a light source to illuminate tissue and output a first wavelength light, a second wavelength light, and white light, wherein both first and second wavelengths are absorbed by oxyhemoglobin and de-oxyhemoglobin while the ratio of the absorption at first and second wavelengths varies with the oxygenation of hemoglobin. The method also includes receiving through a scope the white light, the first wavelength light, and the second wavelength light reflected from the tissue. The method further includes capturing, at one or more cameras coupled to the scope, a plurality of frames of a tissue having a first single wavelength image of the tissue illuminated by the first wavelength light and a second single wavelength image of the tissue illuminated by the second wavelength light. The method further includes generating at an image processing unit, a multispectral image based on the first single wavelength image and the second single wavelength image.

Implementations of the above embodiment may include one or more of the following features. According to one aspect of the above embodiment, the method may also include activating the light emitter intermittently to emit the first wavelength light and the second wavelength light interspersed with the white light. The method may further include replacing, at the image processing unit, the first single wavelength image and the second single wavelength image with a preceding white light image from the plurality of frames and generate a processed video feed. The method may additionally include displaying the processed video feed at a monitor coupled to the image processing unit. The method may also include generating, at the image processing unit, an intensity ratio image based on the first single wavelength image and the second single wavelength image. The method may further include generating, at the image processing unit, the intensity ratio image by calculating an intensity ratio on a pixel-by-pixel ratio between the first single wavelength image and the second single wavelength image. The method may additionally include generating, at the image processing unit, a multispectral image by colorizing the intensity ratio image. The method may also include overlaying the multispectral image over the processed video feed. Activating the light emitter further may include activating a plurality of light emitting diodes (LED) having a first LED configured to emit the first wavelength light, a second LED configured to emit the second wavelength light, and a third LED configured to emit the white light.

According to one embodiment of the present disclosure, an imaging system is disclosed. The imaging system includes a scope having a light port and a view port. The scope is configured to emit light received through the light port and receive the light and provide the light through the view port. The system also includes a multispectral assembly includes a multispectral light assembly coupled to the light port of the scope and configured to emit multispectral light. The multispectral assembly also includes a multispectral camera assembly coupled to the view port of the scope and includes a multispectral camera. The system also includes a light source configured to output white light through the multispectral light assembly to the light port, where the multispectral light assembly is configured to combine the multispectral light and the white light. The system further includes a white light camera coupled to the multispectral camera assembly, where the multispectral camera assembly is configured to split the white light and the multispectral light such that the white light is provided to the white light camera and the multispectral light is provided to the multispectral light assembly.

Implementations of the above embodiment may include one or more of the following features. According to one aspect of the above embodiment, the multispectral light assembly may include a first light source configured to emit a first wavelength light and a second light source configured to emit a second wavelength light at a different wavelength than the first wavelength light, where both first and second wavelengths are absorbed by oxyhemoglobin and de-oxyhemoglobin while the ratio of the absorption at first and second wavelengths varies with the oxygenation of hemoglobin. The multispectral light assembly may further include a first beam splitter configured to combine the first wavelength light and the second wavelength light. The multispectral light assembly may further include a second beam splitter configured to combine the first wavelength light, the second wavelength light, and the white light. The multispectral light assembly may further include a first connector for coupling to the light port and a second connector for coupling to an optical cable of the light source. The first and second connectors are aligned along a straight light path. The second beam splitter may be disposed on the straight light path. The multispectral camera assembly may include a housing having an extension enclosing a camera beam splitter configured to split the white light from the first wavelength light and the second wavelength light.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure may be understood by reference to the accompanying drawings, when considered in conjunction with the subsequent, detailed description, in which:

FIG. 1 is a schematic diagram of an imaging system according to an embodiment of the present disclosure;

FIGS. 2A and 2B are schematic diagrams of an image processing unit according to an embodiment of the present disclosure;

FIG. 3 is a perspective view of a laparoscopic camera according to an embodiment of the present disclosure;

FIG. 4 is a flow chart of a method for multispectral imaging and mapping of tissue oxygen saturation according to an embodiment of the present disclosure;

FIG. 5 is a schematic diagram of light emissions for multispectral imaging using the imaging system of FIG. 1 according to the present disclosure;

FIGS. 6A and 6B are screenshots of images generated by the imaging system according to the present disclosure;

FIG. 7 is a reflectance spectrum of healthy and cancerous colon;

FIG. 8 is a reflectance spectrum of healthy and diverticulitis colon;

FIG. 9 is a Spearman correlation coefficient plot for lean and obese pancreas showing optical correlation corresponding to metabolic disease;

FIG. 10 is a schematic diagram of a multispectral imaging system according to another embodiment of the present disclosure; and FIG. 11 is a schematic diagram of a multispectral imaging system according to a further embodiment of the present disclosure.

### DETAILED DESCRIPTION

Embodiments of the presently disclosed system are described in detail with reference to the drawings, in which like reference numerals designate identical or corresponding elements in each of the several views. In the following description, well-known functions or constructions are not described in detail to avoid obscuring the present disclosure in unnecessary detail. Those skilled in the art will understand that the present disclosure may be adapted for use with any imaging system.

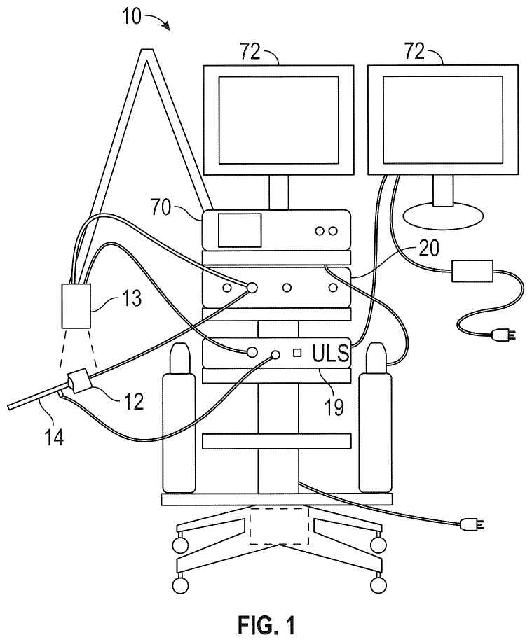

With reference to FIG. 1, an imaging system 10 includes an image processing unit 20 configured to couple to one or more cameras, such as an open surgery camera 13 or an endoscopic camera 12 that is configured to couple to a scope 14, which may be any suitable rigid or flexible medical scope such as a laparoscope, an endoscope, a bronchoscope, a colonoscope, etc. The system 10 also includes a light source 19 coupled to the cameras 12 and 13. The light source 19 may include any suitable light sources, e.g., white light, near infrared, infrared, etc., having light emitting diodes, lamps, lasers, UV light sources, etc. usable with corresponding cameras and sensors disclosed above, as well as UV enhanced cameras.

The image processing unit 20 is configured to receive image data signals from the imaging system 10, process the raw image data from the cameras 12 and 13, and generate blended white light and false colored perfusion, (or other biometric), images for recording and/or real-time display. The image processing unit 20 is also configured to blend images using various AI image augmentations.

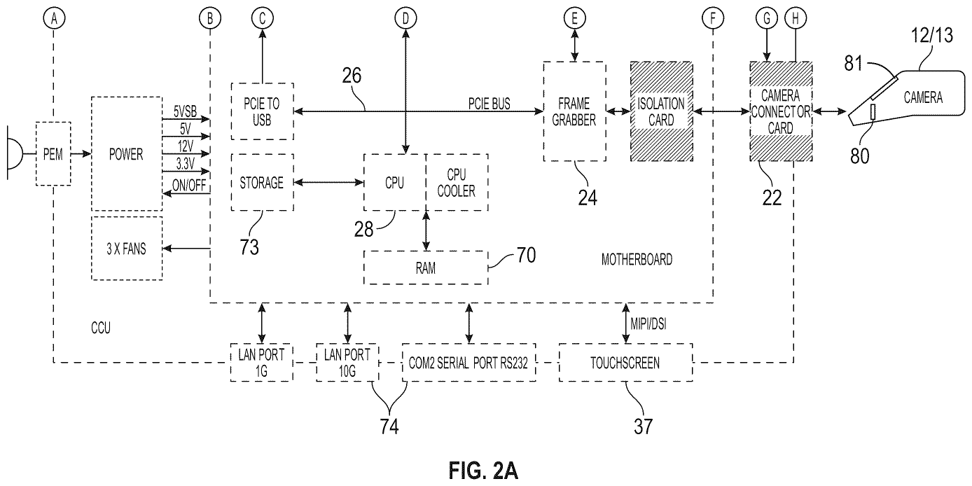

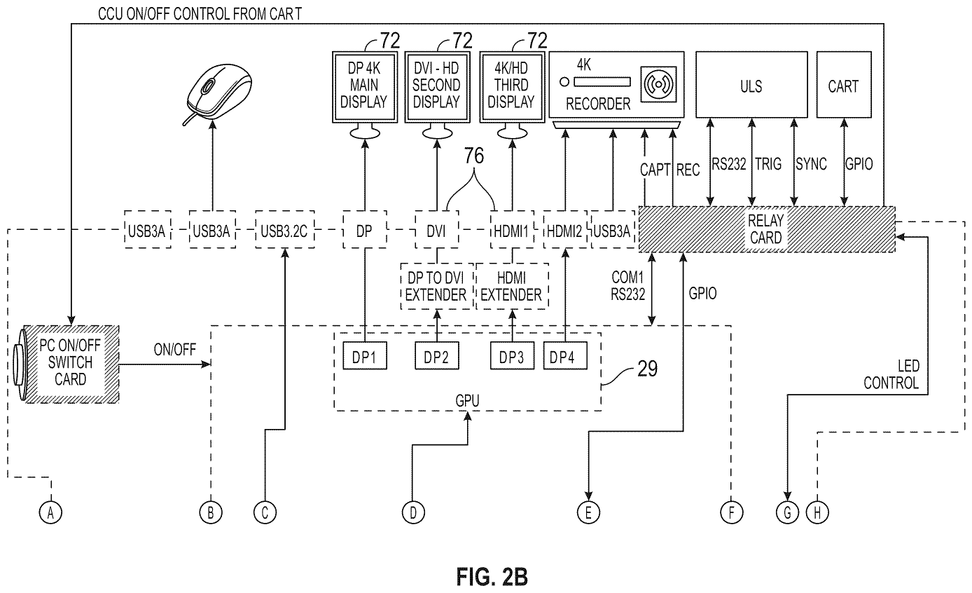

With reference to FIGS. 2A and 2B, the image processing unit 20 is connected to the cameras 12 and 13 through a camera connector 22, which is in turn coupled to a frame grabber 24 that is configured to capture individual, digital still frames from a digital video stream. The frame grabber 24 is coupled via peripheral component interconnect express (PCI-E) bus 26 to a first processing unit 28 and a second processing unit 29. The first processing unit 28 may be configured to perform operations, calculations, and/or sets of instructions described in the disclosure and may be a hardware processor, a field programmable gate array (FPGA), a digital signal processor (DSP), a central processing unit (CPU), a microprocessor, and combinations thereof. Those skilled in the art will appreciate that the processor may be any logic processor (e.g., control circuit) adapted to execute algorithms, calculations, and/or sets of instructions as described herein. The second processing unit 29 may be a graphics processing unit (GPU) or an FPGA, which is capable of more parallel executions than a CPU (e.g., first processing unit 28) due to a larger number of cores, e.g., thousands of compute unified device architecture (CUDA) cores, making it more suitable for processing images.

The image processing unit 20 also includes various other computer components, such as memory 70, a storage device 73, peripheral ports 74, input device (e.g., touch screen). Additionally, the image processing unit 20 is also coupled to one or more monitors 72 via output ports 76. The image processing unit 20 is configured to output the processed images through any suitable video output port, such as a DISPLAYPORT™, HDMI®, SDI, etc., that is capable of transmitting processed images at any desired resolution, display rate, and/or bandwidth.

With continued reference to FIGS. 2A and 2B, the cameras 12 and 13 includes a visible image sensor 80 for white light (i.e., visible light) imaging (e.g., from about 380 nm to about 700 nm) and may also include a separate infrared image sensor 81 (e.g., wavelength from about 750 nm to about 1,700 nm). The image sensor 80 may have any suitable resolution, e.g., 1080p, 4K, etc. The image sensor 80 may include a Bayer filter or any other filter suitable for color single chip imaging. In embodiments, a single sensor may be used to sense white light and IR light, e.g., from about 380 nm to about 1,000 nm, may be used, which may be a monochrome sensor, with or without an IR filter or a single chip color sensor with a Beyer filter. In further embodiments, the cameras 12 and 13 may also use multiple color sensors, e.g., one sensor per color (RGB) channel. In further embodiments, image sensors capable of sensing light having a wavelength from about 300 nm to about 2,000 nm may be used.

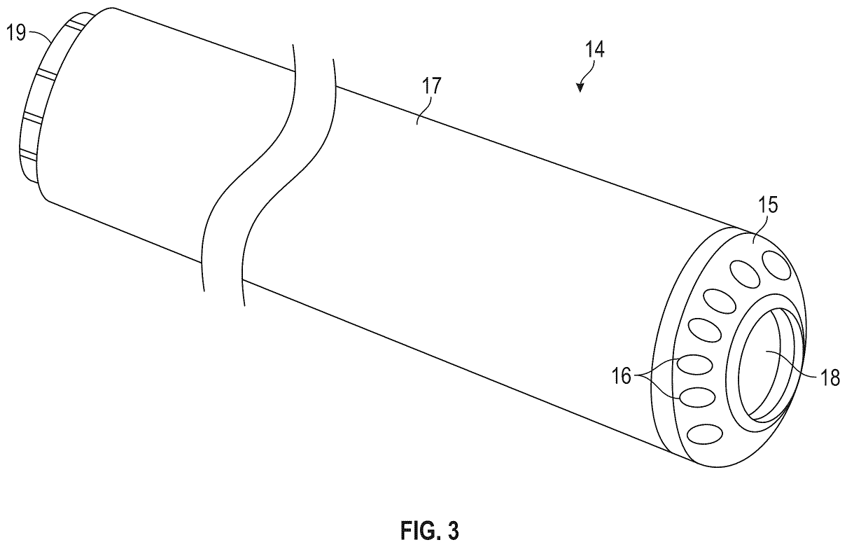

With reference to FIG. 3, the scope 14 may be a monocular or stereoscopic scope having a shaft 17 configured to couple to the camera 12. The scope 14 also includes an objective 18 disposed at a distal end portion of the shaft 17. In embodiments where the scope is stereoscopic individual objectives, one per channel, would be used. The scope 14 may include a multispectral light emitter 15, which acts like the light source 19, and includes a plurality of light emitting diodes (LEDs) 16. In embodiments, the multispectral light emitter 15 may include one or more laser diodes or white light filtered by a monochromator or di-electrical filters or a white-light continuum light source (e.g., a “white laser”). In embodiments, the light emitter 15 at the scope 14 may include various optic elements that transmit light from the light source 19. The light emitter 15 may have a circular shape to provide for efficient placement of the LEDs 16 around the objective 18. In embodiments, the LEDs 16 may be placed in any suitable arrangement relative to the objective 18. One or more of the LEDs 16 is configured to emit white light that is used to image the tissue for visible (i.e., conventional) observation. Each of the LEDs 16 is configured to emit light at a specific wavelength to provide for multispectral imaging, which may be from about 380 nm to about 1,000 nm, and in embodiments, from about 660 nm to about 940 nm. In embodiments, each of the LEDs 16 may be configured to emit light at one of the following wavelengths 540 nm, 560 nm, 580 nm, 660 nm, 720 nm, 770 nm, 810 nm, 860 nm, and 940 nm. In embodiments, the LEDs 16 may be multiwavelength LEDs configured to emit light at multiple wavelengths, thus, minimizing the number of LEDs 16 in the light emitter 15. Furthermore, the multiwavelength LEDs may be used to output white light by combining multiple wavelength bands into the white light. In further embodiments, the light source 19 may include a broad-spectrum light source with filters instead of using discrete LEDs 16.

In another embodiment, the light source 19 may include multiple LEDs 16 configured to emit white light, and light having first and second wavelengths. The light would be transmitted to the scope 14 via optical cable and through the shaft 17 via optical fibers.

The shaft 17 also includes a proximal coupling interface 17a configured to engage the camera 12 with optical elements (not shown) for transmitting return light through the objective 18 as well as electrical contact for powering and controlling the LEDs 16. The scope 14 may include a plurality of lenses, prisms, mirrors, etc. to enable light transmission from the objective 18 to the output elements.

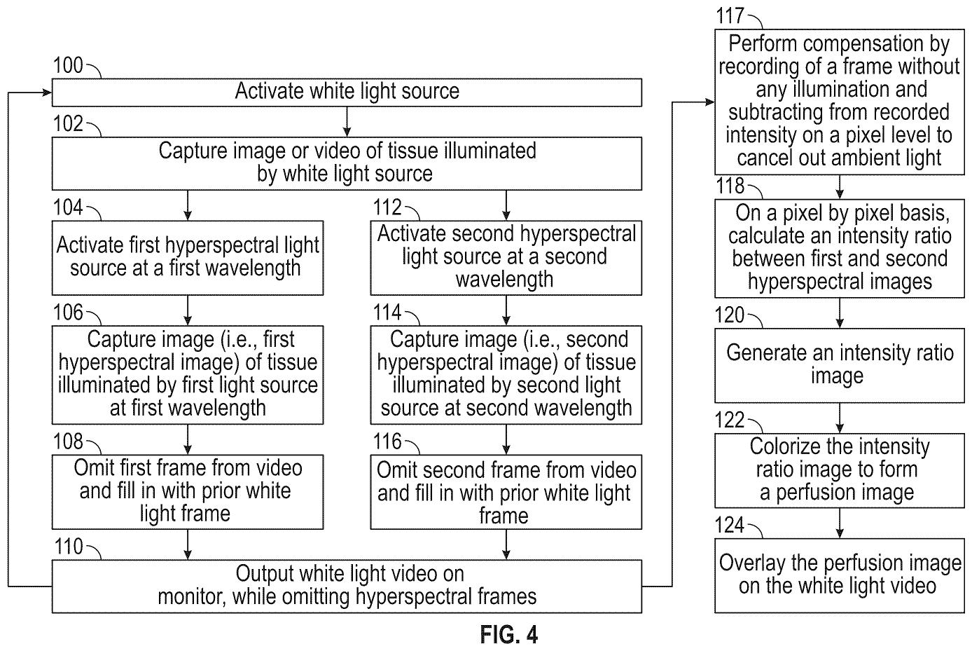

With reference to FIG. 4 a method for multispectral imaging includes at step 100 outputting white light from the light emitter 15 and at step 102 capturing the image or video at the camera 12. The captured image/video is processed by the image processing unit 20 and displayed on one of the monitors 72.

To provide for multispectral imaging, one or more of the frames captured by the camera 12 is a single wavelength frame or image. Specifically, RGB images are obtained on the R/G/B channels, multispectral images are obtained at a small number of discontinuous wavelengths, and hyperspectral images are obtained at a large number of continuous wavelengths. As used herein the term “single wavelength frame” refers to a frame captured by the camera 12 that is illuminated by a light at a specific wavelength, e.g., first and second wavelengths, which may be outside the visible spectrum, and is suitable for imaging oxygenation of tissue, and in particular, with light that is absorbed by oxyhemoglobin and deoxyhemoglobin, respectively. In particular, both first and second wavelengths are absorbed by oxyhemoglobin and de-oxyhemoglobin while the ratio of the absorption at first and second wavelengths varies with the oxygenation of hemoglobin. To accomplish this task, at step 104, the image processing unit 20 switches from white light illumination to a specific single wavelength LEDs 16. In particular, the image processing unit 20 activates the LED 16 that emits light at a first wavelength, which may be a wavelength that is absorbed by deoxyhemoglobin, e.g., from about 540 nm to about 770 nm. At step 106, the camera 12 captures the image of the tissue illuminated by the light at the first wavelength.

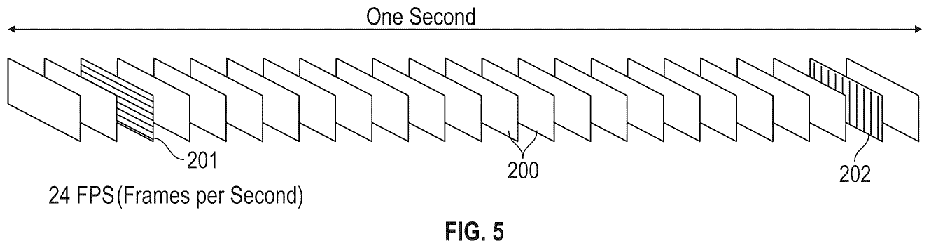

With reference to FIG. 5, an exemplary schematic diagram of a plurality of frames 200 captured by the camera 12 are shown as individual frames and correspond to the refresh rate of the sensor 80, which may be 20 Hz or above. With reference to steps 104 and 106, a first single wavelength frame 201 is included among the plurality of frames 200. The rate at which the LEDs 16 emits the light at the first wavelength, and hence the capture of the first single wavelength frame 201, may be 1/n, where n is the number of frames per second (i.e., refresh rate of the sensor 80 in Hz). During image acquisition, light at each of the selected wavelengths is emitted sequentially with only one wavelength emitted from the LED 16 and synchronized per frame of the camera 12.

The image processing unit 20 outputs white light frames continuously, i.e., live view, on the monitor 72. At step 108, the image processing unit 20 processes the plurality of frames 200 received from the camera 12 and omits and replaces the first single wavelength frame 201 and outputs the frames 200 as a processed video feed on the monitor 72. Since the single wavelength frame 202 was captured under different lighting conditions, inclusion of the first single wavelength frame 201 would result in an inconsistent video stream. In lieu of the omitted first single wavelength frame 201, the image processing unit 20 includes a preceding white color frame of the plurality of frames 200. The preceding frame may be an immediately preceding frame or a few frames prior, depending on the refresh rate of the sensor 80 and/or the monitor 72. At step 110, the image processing unit 20 outputs the processed frames 200 as a video feed on the monitor 72.

The single wavelength illuminated frames may be recorded close to each other (e.g., next to each other or 1-5 white color frames apart) to prevent movement artifacts in the image representing the ratio between said two wavelengths. The displayed frame replacing the single wavelength frame may be a preceding white light frame. In embodiments, the frame may be: White-Wavelength 1-White-Wavelength 2-White, etc.

At steps 112-114, the image processing unit 20 repeats the imaging and illumination of the scene using light at a second wavelength. The sequence of the steps 104-108 and 112-116, i.e., illuminating and imaging tissue using first and second wavelengths, may be switched, such that the tissue is initially illuminated with the second wavelength light and subsequently by the first wavelength light.

At step 112, the image processing unit 20 switches from white light illumination to a specific single wavelength LEDs 16. In particular, the image processing unit 20 activates the LED 16 that emits light at a second wavelength, which may be a wavelength that is absorbed by oxyhemoglobin, e.g., from about 810 nm to about 940 nm. As noted above, both first and second wavelengths are absorbed by oxyhemoglobin and de-oxyhemoglobin while the ratio of the absorption at first and second wavelengths varies with the oxygenation of hemoglobin. At step 114, the camera 12 captures the image of the tissue illuminated by the light at the second wavelength.

With reference to FIG. 5, a second single wavelength frame 202 is included among the plurality of frames 200. The rate at which the LEDs 16 that emits the light at the second wavelength, and hence the capture of the first single wavelength frame 201, may be 1/n, where n is the number of frames per second (i.e., refresh rate of the sensor 80). The first and second single wavelength frames 201 and 202 may be taken any number of frames apart, e.g., 0 or more.

The image processing unit 20 outputs white light frames continuously, i.e., live view, on the monitor 72. At step 116, the image processing unit 20 processes the plurality of frames 200 received from the camera 12 and omits and replaces the second single wavelength frame 202 and outputs the frames 200 as a processed video feed on the monitor 72. Since the second single wavelength frame 202 was captured under different lighting conditions, inclusion of the second single wavelength frame 202 would result in an inconsistent video stream. In lieu of the omitted second single wavelength frame 202, the image processing unit 20 includes a preceding white color frame of the plurality of frames 200. The preceding frame may be an immediately preceding frame or a few frames prior, depending on the refresh rate of the sensor 80 and the monitor 72. At step 110, the image processing unit 20 outputs the processed frames 200 as a video feed on the monitor 72. The steps 100-110 may be continuously looped as the camera 12 is used to image the tissue.

In embodiments, any number of a plurality of wavelengths, i.e., two or more, may be used to generate false color images based on two or more wavelengths. At least one frame or image is obtained at a discrete wavelength and then processed as described below.

While the video captured by the camera 12 and displayed on the monitor 72, the image processing unit 20 is configured to calculate perfusion and oxygen saturation levels based on first and second single wavelength images 201 and 202.

Optionally, the camera 12 may be calibrated prior to its use on a spectrally white reference target and store the results as calibration data, i.e., image. The calibration data may be used by the image processing to compensate the first single wavelength image 201 and the second single wavelength image 202 to improve the uniformity of the mapping and prevent vignetting. Compensation may be performed by applying the inverse of the intensity distribution across the frame using the calibration image as a reference.

At step 117, compensation is performed by recording of a frame without any illumination and subtracting from recorded intensity on a pixel level to cancel out ambient light. Compensation may be performed by applying the inverse of the intensity distribution across the frame as recorded on a spectrally white reference target, which may be provided during calibration of the camera. If ambient light reaches the camera together with the selected single wavelength, compensation adds an offset to the detected intensity. The compensation will affect the ratio between the images formed by first and second wavelength. A mitigation step may also be used to record an image without any light source turned on and then subsequently subtract from frames before compensating for uneven illumination. In this way ambient light will be cancelled out.

At step 118, the image processing unit 20 calculates an intensity ratio on a pixel-by-pixel basis, i.e., for each pixel of the first single wavelength image 201 and a corresponding pixel of the second single wavelength image 202. At step 120, the image processing unit 20 generates an intensity ratio image based on the pixel-by-pixel intensity ratio performed in step 118. At step 122, the image processing unit 20 generates a multispectral image by colorizing the intensity ratio image using one or more any suitable colors, e.g., yellow, red, green, blue, etc., where the intensity of the color corresponds to the calculated intensity ratio. The multispectral image represents perfusion of the tissue based on images of deoxyhemoglobin and oxyhemoglobin, which is s a continuum between one and another.

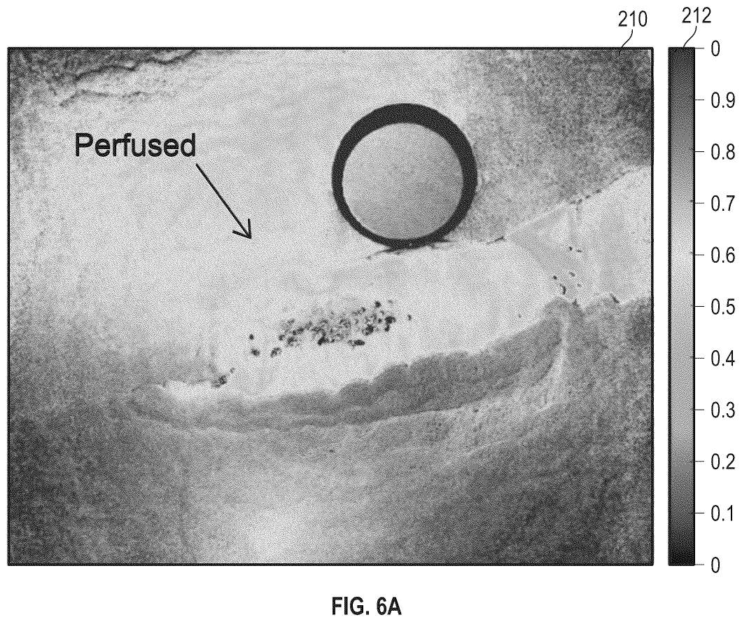

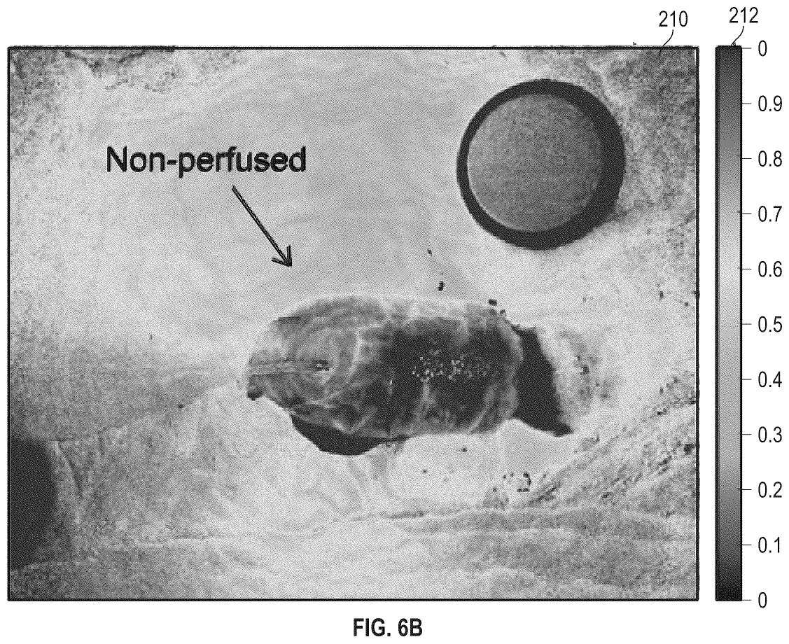

At step 124, the colorized image is overlayed over the white light video feed output at step 110. Exemplary images are shown in FIGS. 6A and 6B with the images 210 and a corresponding heatmap 212.

The imaging system 10 according to the present disclosure may be used in any surgical laparoscopic procedure, and in particular, stapling procedures, such as end-to-end anastomosis procedures in which two portions of a structure (e.g., intestine, colon, etc.) are connected. As noted above, sufficient perfusion is essential to proper healing following a colorectal anastomosis procedure. The imaging system 10 is configured to provide an objective measurement of perfusion and by extension oxygenation, without relying on fluorescence imaging that requires infusion of fluorescent agents, e.g., indocyanine green, in the blood stream, and then comparing relative infrared intensities between perfused and non-perfused areas. The imaging system according to the present disclosure may also be used with robotic surgical systems where camera and instrument positions are controlled to keep track of the mapped tissue area or maybe used along with electromagnetic navigation (i.e., position of camera and instruments to keep track of camera position while recording to achieve same as above).

The system and method of the present disclosure may also be used for multispectral imaging of other compounds and properties of the tissue, such as water, collagen, lipids (e.g., triglycerides), glucose, etc. Water imaging may be used to detect edema and/or inflammation as well as critical structures with different water content than surrounding tissue. Multispectral imaging of water may be performed in short wavelength infrared (SWIR) region. Collagen imaging may be used to diagnose fibrosis and cancer by identifying critical structures in tissue. Multispectral imaging of collagen may be performed in SWIR and NIR regions, i.e., 1000-2000 nm. Lipid multispectral imaging may be used to detect objects with different fat content, e.g., critical structures in obese patients, as well as determine the effect of metabolic diseases on tissue. Multispectral imaging of lipids may be performed in SWIR and NIR regions. Inflammation may be detected based on water content and hemoglobin spectrum due to an increase in fluid and blood. This feature may in turn be used to diagnose Crohn's disease, colitis, diverticulitis, and other conditions. Furthermore, collagen may be imaged using multispectral techniques disclosed herein to quantify the amount of fibrosis. This may be used to identify scaring and radiation and may be used in surgery planning.

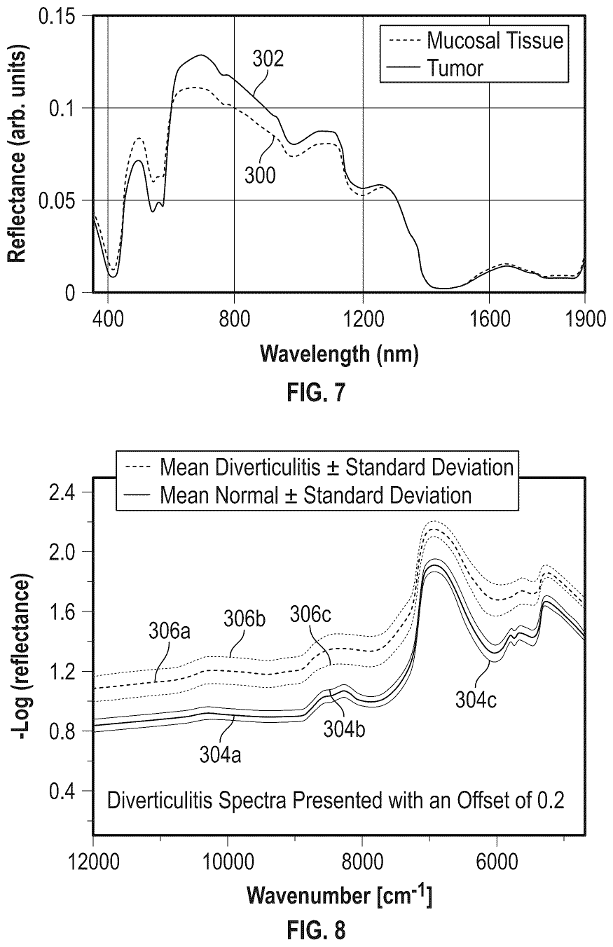

The system may also be used to detect cancer since tumors have a different reflectance spectrum from healthy tissue. FIG. 7 shows the difference between the visible and near-infrared reflectance spectrum for healthy and cancerous colon, i.e., plot 300 shows reflectance of healthy mucosal tissue and plot 302 shows reflectance of a tumor. By measuring the reflectance of tissue at specific wavelengths, the system may be used to differentiate between cancerous and healthy tissue. Highlighting specific wavelengths rather than relying on white light images may be used to intensify the differences. Comparing absolute reflectance differences of single wavelengths or the ratios of different wavelengths is used to generate lookup tables or classifiers for distinguishing healthy and cancerous tissue. In particular, the peak reflectance at around 700 nm provides an absolute reflectance difference between tumor and mucosal tissue. The difference in the ratio of reflectance between 700 nm and 1200 nm may be used to provide a more robust classifying metric as it would account for offsets due to the environment or patient specific differences. While FIG. 7 shows mucosal tissue, the same method may be applied to other tissue types, e.g., small and large bowels.

Multispectral imaging may also be used to detect diverticulitis as illustrated by reflectance plots of healthy colon (plots 304a-c) and a colon having diverticulitis (plots 306a-c) in FIG. 8. Plot 304a shows a population mean reflectance plot for healthy colon and plots 304b and 304c are standard deviation plots of the same. Plot 306a shows a population mean reflectance plot for a colon having diverticulitis and plots 306b and 306c are standard deviation plots of the same. Thus, a similar method may be used to excite the tissue with specific wavelengths (e.g., two or more) that correspond to either absolute reflectance differences or using multiple wavelengths to get ratio differences.

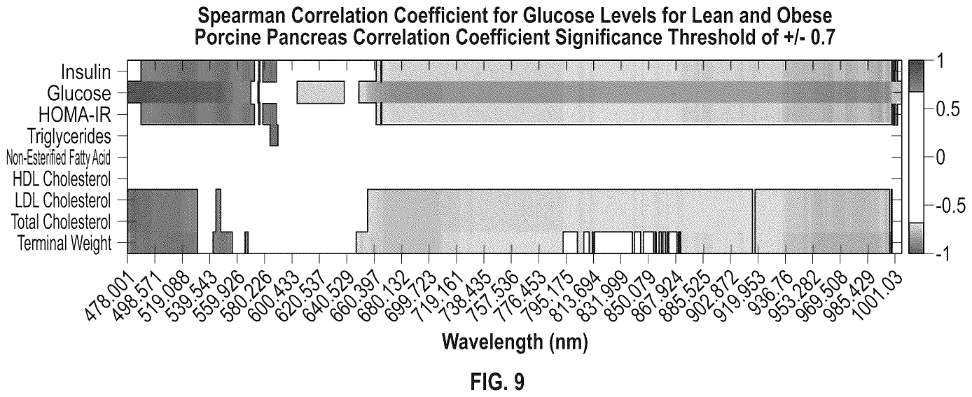

Other molecules in the tissue may also be imaged to detect their concentrations using specific wavelengths as shown in FIG. 9 illustrating optical correlation corresponding to metabolic disease with different biometrics taken from a porcine model where absorption was measured in tissues from 478-1000 nm. Wavelengths in red to dark red show a positive relationship for absorbance for that biometric for a particular wavelength of light. Conversely, green to dark green show a negative correlation for that biometric at a particular wavelength. Glucose absorption may be measured at around 500 nm and 940 nm to create a ratio that increases with increasing glucose levels. This may also be done for all biometrics besides triglycerides, fatty acid, and HDL cholesterol.

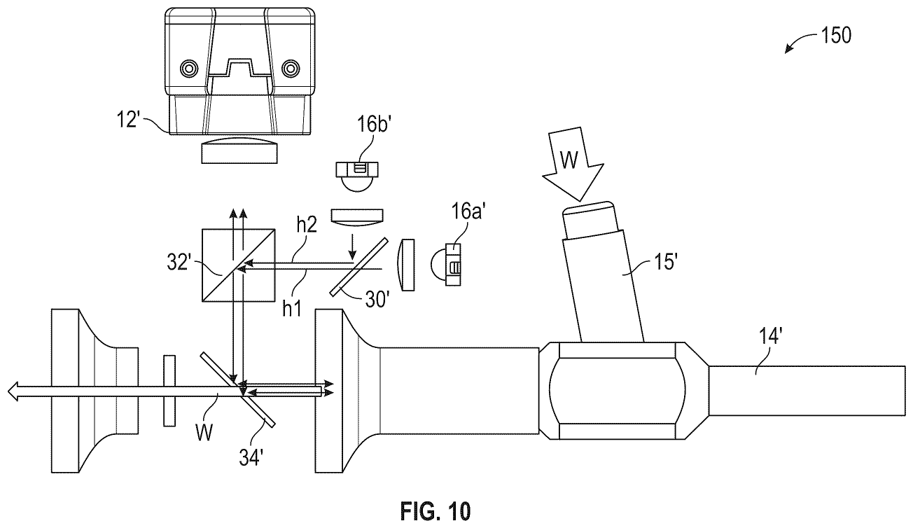

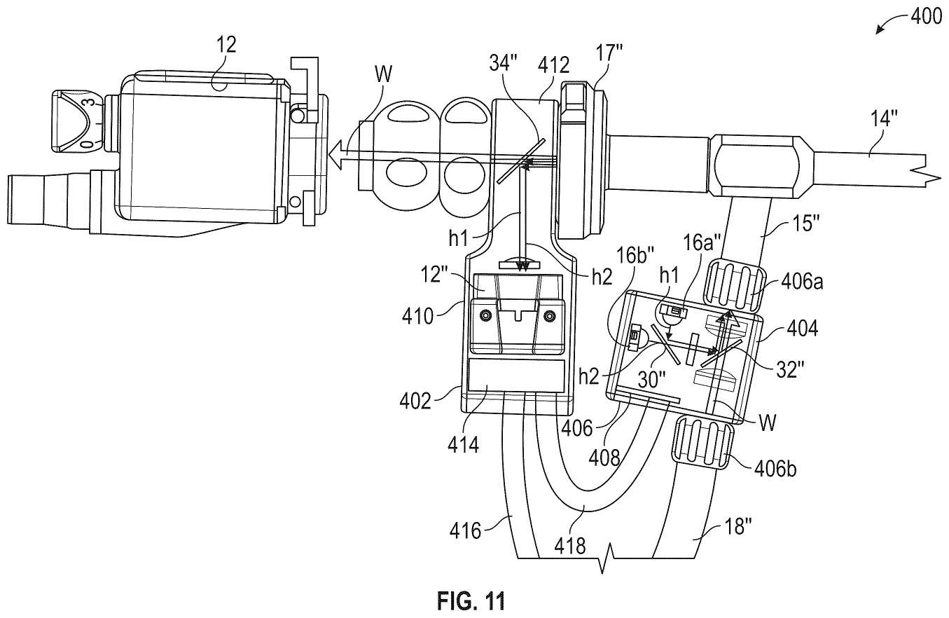

With reference to FIG. 10 another embodiment of an imaging system 150 is shown, which provides for integration of the multispectral light source and camera into the scope 14′. The imaging system 150 may be operated in the same manner as the system 10 and as described above with respect to FIGS. 4-9. In the system 10 the camera 12 is configured to receive light at multiple wavelengths, which is provided by a synchronized light source, i.e., syncing white light and multispectral light sources to the duration and frequency of each frame as imaged by the sensor 80. In contrast, system 150 provides for a dedicated multispectral camera 12′ for added wavelengths beyond the white light imaging provided by the camera 12 and light source 19. The system 150 integrates with the imaging system 10, e.g., light source 19, camera 12, display screens 72, etc. and is a feed-through optical system to the main white light camera 12 and to integrated multispectral camera 12′. The multispectral camera 12′ is also synchronized to the dedicated multispectral light sources, e.g., LEDs 16a′ and 16b′. The integrated light sources are merged with the white light source 19. Using separate cameras 12 and 12′ allows for visualization as separate video feeds on different monitors e.g., where one display screen 72 shows the white light image from the camera 12 and the other display screen 72 shows the multispectral image from the camera 12′. In embodiments, the video feeds may be combined where the multispectral image is overlayed on the white light video feed. Live view may use false color imaging with an optional overlay of low oxygen saturation or perfusion regions.

The multispectral camera setup may also be configured such that the white-light illumination is coupled into the scope 14′ through a light cable port 15′ whereas a multispectral camera 12′ is a feed-through system, which receives the relevant light wavelengths into the scope 14′ through the imaging components (e.g., lenses, prisms, mirrors, etc.) of the scope 14′. A plurality of light sources, e.g., LEDs 16a′ and 16b′ may be used with a corresponding number of dichroic beam splitter(s) 30′ to direct the light to the target through the scope 14′. The LEDs 16a′ and 16b′ may be housed in the light source 19. The number of beam splitters 30′ being used to combine the multispectral light is one less than the total number of LEDs 16′. The first LED 16a′ may emit light at about 660 nm and the second LED 16b′ may emit light at about 940 nm. In embodiments additional LEDs may be used to emit light at different wavelengths.

The beam splitter 30′ predominantly reflects light emitted by one of the LEDs 16′, e.g., LED 16b′ while predominantly transmitting light emitted by the other LED 16′, e.g., first LED 16a′. One or more lenses may be used to collimate or focus the LED beams. A second beam splitter 32′ is used to reflect the light from the LEDs 16a′ and 16b′ into the scope 14′ and transmit the light into the multispectral camera 12′. The beam splitter 32′ may be a non-polarizing beam splitter with a 50 -50 split ratio, but other split ratios and polarizing beam splitters may be used. The beam splitter 32′ receives light from a third beam splitter 34′, which may be a long wavelength reflective dichroic beam splitter that allows visible wavelength light (e.g., up to about 630 nm or higher if an IR camera is being used) to pass through the feed-through setup to be detected by a white light camera that is output on the monitor 72. The visible light may be further filtrated to prevent “red and IR cast” in the surgeon's image displayed on the monitor 72.

White light “w” emitted by a white light source (not shown) is provided through the cable port 15′. The multispectral light at two or more wavelengths h1 (i.e., from LED 16a′) and h2 (i.e., from LED 16b′) is reflected by the first and second beam splitters 30′ and 32′ into the scope 14′. Thus, the white light w1 and the multispectral light h1 and h2 are used to illuminate tissue. The light w, h1, h2 is reflected from the tissue at substantially the same wavelengths, and the reflected white light w passes through the third beam splitter 34′ to be imaged by a white light camera as described above. The beam splitter 34′ reflects most or all of the multispectral light h1 and h2 reflected from the tissue while predominantly transmitting white light w. The reflected multispectral light h1 and h2 then passes through the second beam splitter 32′ to the multispectral camera 12′.

With reference to FIG. 11 another embodiment of an imaging system 400 using a multispectral camera is shown, which is similar to the embodiment of FIG. 10, except that the imaging system 400 transmits all of the light (i.e., white light and multispectral light) through a light port of a scope, obviating the need for beam splitter 32′ used to separate transmitted and reflected light. The imaging system 400 may be operated in the same manner as the systems 10 and 150 and as described above with respect to FIGS. 4-9. The imaging system 400 integrates with the imaging system 10, e.g., light source 19, camera 12, display screens 72, etc. and provides for a dedicated multispectral camera 12″ for added wavelengths beyond the white light imaging provided by the camera 12. The system 400 is a feed-through optical system to the main white light camera 12 and to integrated multispectral camera 12″. The multispectral camera 12″ is also synchronized to the dedicated multispectral light sources, e.g., LEDs 16a″ and 16b″. The integrated light sources are merged with the white light source 19. Using separate cameras 12 and 12″ allows for visualization as separate video feeds on different monitors e.g., where one display screen 72 (see, FIG. 1) shows the white light image from the camera 12 and the other display screen 72 shows the multispectral image from the camera 12″. In embodiments, the video feeds may be combined where the multispectral image is overlayed on the white light video feed. Live view may use false color imaging with an optional overlay of low oxygen saturation or perfusion regions.

The imaging system 400 includes a scope 14″ having a light port 15″ and a proximal coupling interface, i.e., viewport 17″. The imaging system 400 also includes a multispectral camera assembly 402 and a multispectral light source assembly 404. The camera assembly 402 is configured to couple to the viewport 17″ and to the camera 12 (e.g., using one or more zoom lenses) such that the light collected by the scope 14″ passes through the camera assembly 402 and then to the camera 12. Similarly, the light source assembly 404 is coupled to the light port 15″ and to an optical cable 18″ connecting to the light source 19 (FIG. 19).

The light source assembly 404 includes a plurality of light sources, e.g., LEDs 16a″ and 16b″ which may be used with a corresponding number of dichroic beam splitter(s) to direct the light to the target through the scope 14″. The first LED 16a″ may emit light at about 660 nm and the second LED 16b″ may emit light at about 940 nm. In embodiments, additional LEDs may be used to emit light at different wavelengths, such as near infrared wavelength for exciting ICG.

The light source assembly 404 includes a housing 406 enclosing the LEDs 16a″ and 16b″ and other components described below. The multispectral light at first wavelength h1 (i.e., from LED 16a″) hits a first beam splitter 30″, which predominantly reflects light emitted by one of LED 16a″ while predominantly transmitting light at a second wavelength h2 emitted by the LED 16b″.

The housing 406 also includes a first connector 406a for coupling to the port 15″ and a second connector 406b for coupling to the optical cable 18″. The first and second connectors 406a and 406b may be threaded nuts or any other suitable connectors.

In embodiments, the light source assemblymay4 may also be inserted to the white light source 19 with a connector fitting to the white light source 19 at one end, e.g., via the second connector 406b, and the lightguide or optical cable 18″ in the output end, e.g., the first connector 406a. Alternatively, the light source assembly may be inserted in the middle of the light guide or between two lightguides, i.e., the optical cable 18″.

The first and second connectors 406a and 406b are aligned along a main light path, i.e., a straight line, along which the white light w from the optical cable 18″ is transmitted to the port 15″. One or more lenses may be disposed in the housing 406 along the white light w path to collimate or focus the white light w.

A second beam splitter 32″ is also disposed in the housing 406 and along the main light path. The beam splitter 32″ may be a long wavelength reflective dichroic beam splitter that allows visible wavelength light (e.g., up to about 630 nm or higher if an IR camera is being used) to pass through. The beam splitter 32″ also receives and reflects most or all of the multispectral light h1 and h2, such that the three light sources (white light w and multispectral light h1 and h2) are combined for transmission into the light port 15″.

The combined white light w and multispectral light h1 and h2 are shone on the surgical site through the scope 14″. The light is reflected from the surgical site and is received at the scope 14″ as well and passes through the light port 15″ where the camera assembly 402 is attached. The camera assembly 402 includes a housing 410 enclosing a multispectral camera 12″. The housing 410 has an extension 412 with a proximal side and a distal side, where the proximal side is configured to couple to the camera 12 and the distal side is configured to couple to the light port 15″ of the scope 14″. The reflected light passes through the extension 412, which houses a beam splitter 34″ for reflecting most or all of the multispectral light h1 and h2 toward a multispectral camera 12″ while predominantly transmitting white light w toward the camera 12.

The camera assembly 402 also includes a camera control circuit 414 including any suitable processor, memory, etc. for controlling image acquisition and other tasks for operating the multispectral camera 12″. The control circuit 414 may be coupled to the image processing unit 20 via a first cable 416. In addition, the control circuit 414 is also coupled via a second cable 418 to a light control circuit 408 of the light source assembly 404. The light control circuit 408 may include any suitable processor, memory, etc. for controlling operation of the LEDs 16a″ and 16b″.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

1-28. (canceled)

29. An imaging system comprising:
a white-light source configured to output white light to illuminate tissue;
a first-wavelength light source producing light at a first wavelength primarily absorbed by deoxyhemoglobin;
a second-wavelength light source producing light at a second wavelength primarily absorbed by oxyhemoglobin;
a scope configured to receive the white light, the first-wavelength light, and the second-wavelength light reflected from the tissue;
at least one camera coupled to the scope, the at least one camera configured to capture a plurality of frames of the tissue illuminated by at least one of the white light, the first-wavelength light, and the second-wavelength light;
an image processing unit configured to:
activate the white-light source to produce white light to illuminate tissue;
capture frames of a video feed while the tissue is illuminated with the white light;
temporarily deactivate the white-light source at a first time;
while the white-light source is deactivated at the first time:
activate the first-wavelength light source to output the first-wavelength light; and
receive a first single wavelength image of the tissue illuminated by the first-wavelength light;
temporarily deactivate the white-light source at the second time;
while the white light source is deactivated at a second time:
activate the second-wavelength light source to output the second-wavelength light; and
receive a second single wavelength image of the tissue illuminated by the second-wavelength light; and
generate a hyperspectral image based on the first single wavelength image and the second single wavelength image.

30. The imaging system according to claim 29, wherein the image processing unit is further configured to replace the first single wavelength image and the second single wavelength image with a preceding white light image from the plurality of frames and generate a processed video feed.

31. The imaging system according to claim 30, wherein the image processing unit is further configured to generate an intensity ratio image based on the first single wavelength image and the second single wavelength image.

32. The imaging system according to claim 31, wherein the image processing unit is further configured to generate the intensity ratio image by calculating an intensity ratio on a pixel-by-pixel ratio between the first single wavelength image and the second single wavelength image.

33. The imaging system according to claim 31, wherein the image processing unit is further configured to generate the hyperspectral image by colorizing the intensity ratio image.

34. The imaging system according to claim 29, wherein the image processing unit is further configured to overlay the hyperspectral image over the video feed.

35. The imaging system according to claim 29, wherein the first-wavelength light source comprises a first light-emitting diode (LED) and the second-wavelength light source comprises a second LED.

36. The imaging system according to claim 29, wherein the white-light source, the first-wavelength light source, and the second-wavelength light source are disposed at a distal end portion of the scope.

37. The imaging system according to claim 29, further comprising:
a first dichroic beam splitter configured to combine the first and second wavelength lights for transmission through the scope to illuminate the tissue.

38. The imaging system according to claim 37, further comprising:
a second dichroic beam splitter configured to:
reflect the first and second wavelength lights for transmission through the scope to illuminate the tissue; and
reflect the first and second wavelength lights reflected from the tissue to the at least one camera and transmit white light to another camera.

39. The system according to claim 29, wherein the image processing unit is further configured to receive a calibration image of a spectrally white reference target.

40. The system according to claim 39, wherein the image processing unit is further configured to process at least one of the first single wavelength image or the second single wavelength image to compensate for or prevent vignetting.

41. The system according to claim 39, wherein the image processing unit is further configured to apply an inverse of an intensity distribution across at least one of the first single wavelength image or the second single wavelength image using the calibration image.

42. A method for imaging perfusion of tissue, the method comprising:
activating a white-light source to illuminate tissue;
receiving through a scope the white light reflected from the tissue;
capturing, at a camera coupled to the scope, a plurality of frames of the tissue for a video feed;
temporarily deactivating the white-light source at a first time;
while the white-light source is deactivated at the first time:
activating a first-wavelength light source to illuminate the tissue with a first-wavelength light; and
capturing, with the camera, a first-wavelength frame of the tissue while the tissue is illuminated with the first-wavelength light;
temporarily deactivating the white-light source at a second time;
while the white-light source is deactivated at the second time:
activating a second-wavelength light source to illuminate the tissue with a second-wavelength light; and
capturing, with the camera, a second-wavelength frame of the tissue while the tissue is illuminated with the second-wavelength light; and
generating, at an image processing unit, a hyperspectral image based on the first-wavelength frame and the second-wavelength frame.

43. The method according to claim 42, further comprising replacing, at the image processing unit, the first-wavelength frame and the second-wavelength frame with a preceding white-light frame from the plurality of frames and generating a processed video feed.

44. The method according to claim 43, further comprising displaying the processed video feed at a monitor coupled to the image processing unit.

45. The method according to claim 42, further comprising generating, at the image processing unit, an intensity ratio image based on the first-wavelength frame and the second-wavelength frame.

46. The method according to claim 45, further comprising:
generating, at the image processing unit, the intensity ratio image by calculating an intensity ratio on a pixel-by-pixel basis between the first-wavelength frame and the second-wavelength frame.

47. The method according to claim 45, further comprising:
generating, at the image processing unit, the hyperspectral image by colorizing the intensity ratio image.

48. The method according to claim 43, further comprising overlaying the hyperspectral image over the processed video feed.
